(19) United States
(12) Patent Application Publication
(10) Pub. No.: US 2014/0100385 A1
(43) Pub. Date: Apr. 10, 2014

(54) PROCESS FOR PREPARATION OF SUCCINYLCHOLINE CHLORIDE

(71) Applicant: SEQUENT SCIENTIFIC LIMITED, Mumbai (IN)

(72) Inventors: Mahalinga MANJATHURU, Mangalore (IN); Anil Narayan MAYEKAR, Mangalore (IN); Pejakala Kakrannaya VASUDEVA, Mangalore (IN); Thangavel ARULMOLI, Bangalore (IN); Gautam Kumar DAS, Bangalore (IN)

(73) Assignee: SEQUENT SCIENTIFIC LIMITED, Mumbai (IN)

(21) Appl. No.: 13/771,872

(22) Filed: Feb. 20, 2013

(30) Foreign Application Priority Data
Oct. 8, 2012 (IN) 2938/MUM/2012

(51) Int. Cl. C07C 213/06

(52) U.S. Cl. CPC C07C 213/06
USPC 560/196

(57) ABSTRACT

The present invention relates to a process for the preparation of succinylcholine chloride, a pharmaceutically active compound used as skeletal muscle relaxant which comprises condensing succinic anhydride with N,N-diemthylaminoethanol using a catalyst in presence of a solvent to form bis[2-(dimethylamino)ethyl]succinate; in situ purifying the bis[2-(dimethylamino)ethyl]succinate using a base; reacting pure bis[2-(dimethylamino)ethyl]succinate with methyl chloride gas using an alcohol; and purifying the obtained crude succinylcholine chloride using water and alcohol.

This application claims the priority of Indian Patent Application No. 2938/MUM/2012, filed Oct. 8, 2012, which is incorporated herein by reference.

### FIELD OF INVENTION

The present invention relates to a novel process for the preparation of Succinylcholine Chloride, a pharmaceutically active compound used as skeletal muscle relaxant.

### BACKGROUND OF THE INVENTION

A Muscle Relaxant is a drug that causes skeletal muscle contraction to cease. Muscle relaxants are used to facilitate surgery, to enable tracheal intubations and to facilitate mechanical ventilation. Muscles relaxants typically work by blocking the effect of acetylcholine (ACh) at the neuromuscular junction.

Substances that compete with ACh, for the receptors on a muscle cell can be either depolarizing, or non-depolarizing. Depolarizing muscle relaxants activate the muscle briefly, before blocking it; e.g. Succinyl choline or Suxamethonium non-depolarizing relaxants block the ACh receptors without activating them, whereas curare, or curare-based molecules, such as Tubocurarine, Pancuronium bromide, Mivacurium, Atracurium, Cisatracurium, Vecuronium, and Rocoronium skeletal muscle relaxants, are drugs that relax striated muscles (those that control the skeleton).

The prolonged stimulation of the acetylcholine receptor results first in disorganized muscle contractions, then in profound relaxation. Its medical uses are limited to short-term muscle relaxation in anaesthesia and intensive care, usually for facilitation of tracheal intubations. Despite its many undesired effects on the circulatory system and skeletal muscles (including malignant hyperthermia, a rare but life-threatening disease), it is still used, because it arguably has the fastest Mad of action of all muscle relaxants.

Succinylcholine chloride is sold under the trade names Anectine, Quelicin, and Scoline. The chemical name of succinylcholine chloride is 2,2′-[(1,4-dioxobutane-1,4-diyl)bis(oxy)]bis (N,N,N-trimethylethanaminium) chloride represented by formula I,

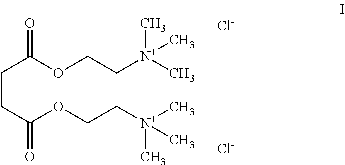 [SMILES: C[N+](C)(C)CCOC(=O)CCC(=O)OCC[N+](C)(C)C ; [Cl-] ; [Cl-]]

There are a few processes reported for the preparation of this compound. U.S. Pat. No. 2,858,329 describes a process for the preparation of bis-dimethylaminoethylsuccinate by heating succinic anhydride and N,N-dimethylaminoethanol at 135-145° C. in benzene for 12-18 hr. The obtained succinylcholine is further purified by high vacuum distillation at very high temperature. The obtained succinylcholine is then purged with molar equivalent of methylchloride in isopropyl alcohol (IPA) and water at 40-50° C. to obtain succinylcholine chloride.

U.S. Pat. No. 5,206,420 describes a process for the preparation of succinyl choline halide. According to this patent, dialkyl succinate is reacted with large excess of dimethyl amino ethanol, in presence of a base catalyst viz. alkali metal alcoholate or amide as catalyst. The bis(2-dimethylaminoethyl) succinate obtained is then reacted with methyl halide in an inert solvent to yield succinyl choline halide.

The prior art processes involve purification of bis-dimethylaminoethyl succinate using high vacuum distillation at very high temperature. Since the product is highly heat sensitive, substantial portion is decomposed during high vacuum distillation. Those processes need special heating facility, which is not desired at industrial level. The high vacuum distillation further poses a safety risk. The prior art reaction is carried out in benzene which is carcinogenic, and thus, is not desired at industrial level. Moreover, the recovery of IPA used from the reaction is cumbersome.

Thus, there remains a need to develop a process that overcomes the drawbacks of the prior art. The present inventors have developed a very cost effective and environment friendly process, which overcomes most of the above stated drawbacks.

### SUMMARY OF THE INVENTION

The present invention relates to the process illustrated by reactions in Scheme 1:

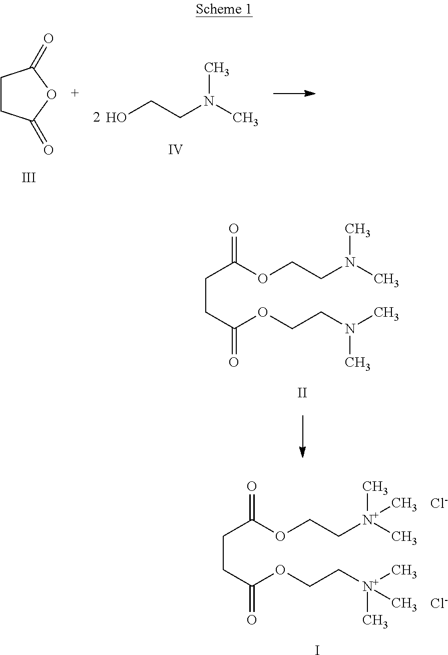

The principal aspect of the present invention is to provide a process for the preparation of 2,2′-[(1,4-dioxobutane-1,4-diyl)bis(oxy)]bis (N,N,N-trimethylethanaminium) chloride represented by formula 1, which comprises:
a) condensing succinic anhydride of formula III with N,N-dimethylaminoethanol of formula IV using an acid catalyst or a catalytic quantity of acidic resin in presence of a solvent to form bis[2-(dimethylamino)ethyl] succinate of formula II;
b) purifying, the bis[2-(dimethylamino)ethyl] succinate of formula II using a base;
c) reacting the obtained bis[2-dimethylamino)ethyl] succinate of formula II with methyl chloride gas in a solvent to obtain 2,2′-[(1,4-dioxobutane-1,4-diyl)bis(oxy)]bis (N,N,N-trimethylethanaminium) of formula I; and
d) purifying the obtained crude 2,2′-[(1,4-dioxobutane-1,4-diyl)bis(oxy)]bis (N,N,N-trimethylethanaminium) of formula I using water and alcohol.

### DETAIL DESCRIPTION OF THE INVENTION

Accordingly in an embodiment of the invention, the condensation succinic anhydride (formula III) with N,N-Diemthylaminoethanol (formula IV) (step a)) is carried out using an acid catalyst or as catalytic amount of acidic resin. The acid catalyst can be, but is not limited to, mineral acids, trifluoro acetic acid, or p-toluene sulfonic acid (PTSA). The mineral acid is selected from sulphuric acid, phosphoric acid, and hydrochloric acid. The acid resin is preferably an acidic resins viz. amberlites. Most preferably, the catalyst used is p-toluene sulfonic acid.

The solvent for the condensation reaction in step a) is an inert organic solvent, such as toluene, benzene, chlorobenzene, xylene and the like, with toluene being the preferred solvent. The reaction is preferably carried out at reflux temperature in the range of about 100-130° C., more preferably about 110-120° C. The reaction produces bis[2-(dimethylamino)ethyl]succinate (formula II).

In another embodiment of the invention, the base for the purification of bis[2-(dimethylamino)ethyl]succinate (formula II) is an alkali metal carbonate or bicarbonate, such as sodium carbonate, potassium carbonate, sodium bicarbonate, potassium bicarbonate and the like, or an aliphatic organic base, such as triethylamine and diisopropylamine, or an aromatic organic base, such as pyridine, picoline, and the like. The preferred base for the purification is potassium carbonate.

In still another embodiment of the invention, the toluene layer, containing bis[2-(dimethylamino)ethyl]succinate (formula II) obtained in step (b) after purification with a base, may be taken for the next step directly without distillation of the solvent. This avoids the need of other solvents for the quaternisation reaction with methyl chloride gas.

In another embodiment of the invention, the reaction of bis[2-(dimethylamino)ethyl]succinate (formula II) with methyl chloride gas in step (c) is carried out using a solvent, such as methanol, ethanol, propanol, butanol, isopropanol, acetone and toluene. The preferred solvent is isopropanol. The reaction preferably takes place at temperature of about 55-65° C., more preferably 60-62° C.

In another embodiment of the invention, the purification of crude 2,2′-[(1,4-dioxobutane-1,4-diyl)bis(oxy)]bis (N,N,N-trimethylethanaminium) (formula I) is accomplished using water and alcohol, such as methanol, ethanol, propanol, butanol, isopropanol and the like. The preferred alcohol is isopropanol. The crude 2,2-[(1,4-dioxobutane-1,4-diyl)bis(oxy)]bis (N,N,N-trimethylethanaminium) (formula I) may be extracted in water without isolation and subjected to alcohol water purification.

Some of the key advantages of the present invention are:
1. In the process of the present invention purification of bis-dimethylaminoethylsuccinate is done by a simple base avoiding high vacuum distillation which leads to decomposition of the product.
2. The process of the present invention doesn't require special heating facility to obtain pure bis-dimethylaminoethylsuccinate of purity more than 99%.
3. In the process of the present invention, distillation of solvent is avoided and the same solvent layer is subjected to quaternisation reaction obviating the need of other solvent.
4. The process of the present invention is carried out in presence of catalyst p-toluene sulfonic acid, which reduces the reaction time significantly.
5. The reaction of bis-dimethylaminoethylsuccinate with methyl chloride gas is carried out in presence of IPA instead IPA water mixture, which makes the recovery of IPA very easy.
6. In the process of the present invention, the crude succinylcholine chloride is extracted in water and subjected to purification without isolation of the crude.

Without further description, it is believed that one of ordinary skill in the art can, using the preceding description and the following illustrative examples, make and utilize the compounds of the present invention and practice the claimed methods. The following examples are given to illustrate the present invention. It should be understood that the invention is not to be limited to the specific conditions or details described in the examples.

### EXAMPLE 1

### Preparation of 2,2′[(1,4-dioxobutane-1,4-diyl)bis(oxy)]bis (N,N,N-trimethylethanaminium):

(a) Procedure for the Preparation of Bisdimethylaminoethyl succinate

Succinic anhydride (0.756 kg), N,N-Diemthylaminoethanol (1.483 kg), toluene (3.78 L) and p-toluene sulfonic acid (0.0189 kg) were charged into a flask at 30±2° C., heated to reflux (˜120° C.) for 2 hours with stirring, and maintained for 30-35 hours. The water was removed by azeotropic distillation. After completion of the reaction, the reaction mass was cooled to 30±2° C. A potassium carbonate solution (0.378 kg of potassium carbonate in 0.378 L of demineralized (DM) water) was charged into the above reaction mass, stirred for 30 minutes, and allowed to settle for 1 hour. The layers were separated. Toluene was distilled out completely under vacuum below 75° C. and the material was unloaded.

Yield: 80%

Purity: 99.5%

(b) Procedure for the Preparation of succinylcholine chloride

Isopropyl alcohol (IPA) (12.48 L) and bis-dimethylaminoethylsuccinate (1.04 kg) were charged into an autoclave at 30±2° C. Methyl chloride gas was purged to the reaction mass at 30±2° C. with stirring under 1.5 to 2.5 kg/cm² pressure, stirred for 15 minutes, heated to 60±2° C. for 16 hours, and cooled to 30±2° C. After completion of the reaction, it was cooled to 7±2° C. and stirred for 1 hour. The reaction mass was washed with IPA (2.08 L) and vacuum dried.

Yield: 92%

Purity: 97 to 98%

(c) Procedure for the Purification of crude sucinylcholine chloride

Crude succinylcholine chloride (1.29 kg) was dissolved in DM water (1.29L) by heating to 50±2° C. Activated charcoal (0.0645 kg) was charged and stirred at 50±2° C. for 1 hour under stirring. The reaction mass was filtered through a hyflo supercell bed, washed with hot DM water. The filtrate was cooled to 28±2° C. The filtrate was charged into IPA (11.61 L), stirred, and heated to 80±2° C. The reaction mass was cooled under stirring to obtain a crystalline sold and maintained for 2 hours at 7±2° C. The solid was filtered, washed with chilled IPA, dried for 2 hours under vacuum.

Yield: 90%

Purity: 99.5%

### EXAMPLE 2

### Preparation of 2,2′-[(1,4-dioxobutane-1,4-diyl)bis(oxy)]bis (N,N,N-trimethylethanaminium)

(a) Procedure for File Preparation of Bisdimethylaminoethyl succinate

Succinic anhydride (0.756 kg), N,N-Diemthylaminoethanol (1.483 kg), toluene (3.78 L) and p-toluene sulfonic acid (0.0189 kg) were charged into a flask at 30±2° C., heated to reflux (˜120° C.) for 2 hours with stirring, and maintained for 30-35 hours. The water was removed by azeotropic distillation. After completion of the reaction, the reaction mass was cooled to 30±2° C. A potassium carbonate solution (0.378 kg of potassium carbonate in 0.378 L of DM water) was charged into the above reaction mass, stirred for 30 minutes, and allowed to settle for 1 hour. The layers were separated; and the toluene layer was collected.

(b) Procedure for the Preparation of succinylcholine chloride

The above toluene layer containing bis-dimethylaminoethyl succinate was charged into an autoclave at 30±2° C. Methyl chloride gas was purged to the reaction mass with stirring under 1.5 to 2.5 kg/cm² pressure, stirred for 15 minutes, heated to 60±2° C. for 16 hours, and cooled to 30±2° C. After completion of the reaction, it was cooled to 25-30° C. The product was extracted in 1.6 L of water. Activated charcoal (0.05 kg) was charged to the extracted mass and stirred at 50±2° C. for 1 hour under stirring. The reaction mass was filtered through a hyflo supercell bed, washed with hot water. The filtrate was cooled to 28±2° C. The filtrate was then charged into IPA (14.4 L), stirred, and heated to 80±2° C., The reaction mass was cooled under stirring to obtain a crystalline solid and maintained for 2 hours at 7±2° C. The solid was filtered, washed with chilled IPA, dried for 2 hours under vacuum.

Yield: 88%

Purity: 99.5%

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

We claim:

1. A process for the preparation of 2,2′-[(1,4-dioxobutane-1,4-diyl)bis(oxy)]bis (N,N,N-trimethylethanaminium) chloride of formula I 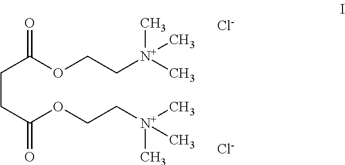 [SMILES: C[N+](C)(C)CCOC(=O)CCC(=O)OCC[N+](C)(C)C ; [Cl-] ; [Cl-]]
comprising the steps of:
a) condensing succinic anhydride of formula III with N,N-dimethylaminoethanol of formula IV, using an acid catalyst or a catalytic amount of acidic resin in presence of a solvent, to form bis[2-(dimethylamino)ethyl]succinate of formula II; 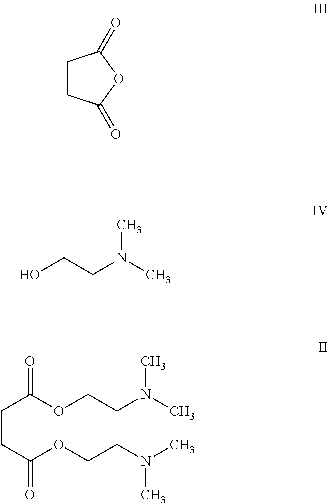
b) purifying the bis[2-(dimethylamino)ethyl]succinate of formula II using a base;
c) reacting the bis[2-(dimethylamino)ethyl]succinate of formula II obtained in step b) with methyl chloride gas to obtain crude 2,2′-[(1,4-dioxobutane-1,4-diyl)bis(oxy)]bis (N,N,N-trimethylethanaminium) of formula I; and
d) purifying the crude 2,2′-[(1,4-dioxobutane-1,4-diyl)bis(oxy)]bis (N,N,N-trimethylethanaminium) of formula I using water and alcohol.

2. The process of claim 1, wherein the catalyst in step a) is selected from the group consisting of mineral acids, trifluoro acetic acid, and p-toluene sulfonic acid (PTSA).

3. The process of claim 1, wherein the acid catalyst in step a) is p-toluene sulfonic acid.

4. The process of claim 1, wherein the acidic resin is an amberlite.

5. The process of claim 1, wherein the solvent in step a) is an inert organic solvent selected from the group consisting of toluene, benzene, and xylene.

6. The process of claim 1, wherein the solvent in step a) is toluene.

7. The process of claim 1, wherein the condensation in step a) is carried out at about 110-120° C.

8. The process of claim 1, wherein the base used in step b) is an alkali metal carbonate or alkali metal bicarbonate or an organic base.

9. The process of claim 1, wherein the base used in step b) is potassium carbonate.

10. The process of claim 1, wherein the reaction in step c) is carried out at about 55-65° C.

11. The process of claim 1, wherein step c) occurs in a solvent.

12. The process of claim 11, wherein the solvent is isopropanol.

13. The process of claim 1, the alcohol in step d) is isopropanol.
